(19) United States
(12) Patent Application Publication
(10) Pub. No.: US 2005/0055062 A1
(43) Pub. Date: Mar. 10, 2005

(54) Screwless system for connecting a probe to an active implantable medical device

(76) Inventors: Philippe Correas, Suisy-Sous Montmoreney (FR); Pascal Vanhee, Draveil (FR)

Correspondence Address: Orrick, Herrington & Sutcliffe LLP, 666 Fifth Avenue, New York, NY, 10103, US

(21) Appl. No.: 10/912,778

(22) Filed: Aug. 4, 2004

(30) Foreign Application Priority Data
Aug. 5, 2003 (FR) 03-09630

(51) Int. Cl. A61N 1/375

(52) U.S. Cl. 607/37

(57) ABSTRACT

A screwless system for connecting a probe to an active implantable medical device. The connector of the generator has an axial female housing (16) able to receive a probe connector (18), and a reversible mechanical retention system to secure the probe connector (18) in the housing (16). The system uses a rotary bolt (14) equipped with a side cam surface (30), defined by a curvilinear section whose radius compared to the axis of rotation is a variable radius having different semicylindrical sections, and is provided with a drive means structure (50) that can be driven by an external tool to rotate the bolt in a housing (20) around an axis (22) between a retracted position, where the bolt cam surface leaves free the interior volume of the female housing so as to freely authorize the movement of the probe connector in the female housing, and a locking position where the cam surface protrudes inside the female housing so as to engage and cooperate locally with an external surface of the probe connector to exert a radially directed force, and thereby tighten the probe connector inside the female housing.

### FIELD OF THE INVENTION

The present invention relates to active implantable medical devices and it will be described in the context of a cardiac pacemaker. However, it should be understood that this is only one example of an embodiment of the invention, and the invention is applicable in a much more general manner to a wide variety of “active implantable medical devices” as those devices are defined by the Jun. 20, 1990 Directive 90/385/CE of the Council of the European Communities. That definition includes in addition to cardiac pacemakers devices such as defibrillators and/or cardiovertors, neurological apparatuses, pumps for diffusion of medical substances, cochlear implants, implanted biological sensors, etc.

### BACKGROUND OF THE INVENTION

Active implantable medical devices, such as cardiac pacemakers, generally have a case designated as a generator or pulse generator, that is connected electrically and mechanically to a probe, with the connection being realized by the surgeon at the time of the implantation.

Reference is made to the French and European standard NF EN 50077, titled “Low profile connector for implantable cardiac pacemakers,” which defines a system of standardized connections known as “IS-1,” making it possible to guarantee the interchangeability of the probes and the pulse generators produced by various manufacturers. It should be understood, however, that the invention is not limited to the particular embodiment of the connection system according to this standard, as it is not limited to connection systems for cardiac pacemakers.

Generally, the connector of the generator includes one or more connection terminals that receives the probe. For each connection terminal, a metal part called an “insert” is provided, which is a part comprising an axial female housing for receiving a male part of the probe called the “connector” (or “probe connector”) at the place where the contact must be established with a conducting area of the probe connector. The insert is electrically connected to the circuit of the generator by a conducting wire or pin and it is encased, at the same time as the other inserts of the generator connector, in a bio-compatible material forming the “connector head” of the generator.

The electric and mechanical connection of the probe connector to the generator connector is generally carried out by tightening a screw that is provided with the insert, mounted transversely to the axial female housing, so that the screw advances and protrudes into the axial female housing receiving the probe connector to engage the probe connector. The various screws for the different inserts are tightened by the surgeon using an appropriate tool (e.g., a screwdriver, possibly provided with a torque limiter) at the time of the implantation.

The known system using that transverse screw presents, however, several disadvantages. First, during the preliminary manipulations for fixing the probe connector, the screws can leave the insert or, conversely, can fall into the cavity of the insert. Second, at moment of tightening of the screw, there exists a risk of damaging the sealing plugs provided for (i.e., the plugs that come to protect the screw heads from the contact with the organic fluids after implantation) when using the tool. Third, during the production of the device, it is necessary to provide for a specific stage for the installation of the screws in the inserts. Fourth, to take account of the axial travel of the screw, it is necessary to provide a sufficient space in the connector, with a correlative increase in the size (volume) of the connector head.

Various alternative probe connectors systems have been proposed to overcome these disadvantages that do not use the tightening of screws, such as are described, for example, in U.S. Pat. No. 5,951,595 or U.S. Pat. No. 6,080,188. These connecting systems, if they are effective from the mechanical and electric point of view, nevertheless imply a significant complexity of structure. There is a multiplication of the moving parts required to carry out precise and low-profile fittings, and these various constraints imply a significant increased cost of manufacture.

EP-A-0 900 577 also describes a screwless connecting system, but the proposed aim of this document is to have a system that provides for a fast-locking connection, without use of any tool and with a “non-return” function preventing an accidental withdrawal of the probe connector. The mechanical tightening obtained by the auto-locking system is however quite less than that of the traditional systems with a screw and the screwless systems as suggested by the U.S. Pat. No. 5,951,595 or U.S. Pat. No. 6,080,188 mentioned above. Moreover, the need for envisaging an auto-locking system implies a particular geometry of the system to obtain the required non-return effect, with a swiveling part pulled by a spring in the opposite direction of insertion.

### OBJECTS AND SUMMARY OF THE INVENTION

The present invention has as an object to mitigate the disadvantages of the systems previously suggested, by application of a mechanical system for retaining the probe connector that is of a particularly simple and effective structure, and that makes it possible to obtain the same mechanical performance as in a system requiring tightening of a screw, but while avoiding using such screws and the associated disadvantages noted above.

To this end, the present invention is broadly directed to an active implantable medical device having a generator connector comprising a housing body containing an axial female housing able to receive a probe connector, and a reversible means for mechanical retention of the probe connector in the housing body. The reversible means for mechanical retention is characterized by a rotary bolt having an axis of rotation and equipped with a side cam surface, and a drive means structure. The drive means structure has a shape that is complementary to an external tool that couples to the structure and is operated to exert on the bolt a rotational driving couple (force) around its axis of rotation. The bolt thus can be moved from a retracted position, where the bolt leaves free (i.e., does not protrude into) the interior volume of the female housing so as to freely permit the insertion of the probe connector in the female housing, to a locking position, where the side cam surface of the bolt protrudes inside the female housing so as to engage and cooperate locally with an external surface of the probe connector to exert on the probe connector a force, radially directed, for locking and mechanically coupling the probe connector secure inside the female housing. The bolt also can be moved from the locking position to the retracted position for removal of the probe connector, and, e.g., replacement of the implant, the probe or both.

In a preferred embodiment, the side cam surface of the rotary bolt is a surface defined by a curvilinear section having a variable radius relative to its axis of rotation. More preferably, the curvilinear section includes two semicircular portions, one portion having a relatively smaller diameter centered on the axis of rotation of the rotary bolt, and the other portion having a larger diameter centered on an axis rotation that is offset relative to the axis of rotation of the rotary bolt. In this latter embodiment, the two portions are connected in continuity at one of their extremities (i.e., a smooth surface transition between the two portions), whereas at their other extremities, the two semicircular portions are connected by forming a discontinuity or a step. The discontinuity is advantageously used as a rotational travel stop by cooperating with a bump homologous on the body of the connector insert.

In a particular preferred embodiment, the aforementioned drive means structure complementary to an external tool is configured as a hollow stamp or shaped cavity, formed axially in the rotary bolt, such as a polygonal cavity that can receive the complementary shaped drive end of an external tool.

Preferably, the rotary bolt comprises, at one and/or the other of its axial extremities, an external cylindrical surface cooperating with an internal cylindrical surface of the housing body receiving the rotary bolt, essentially of the same diameter to allow guidance in rotation of the rotary bolt around its rotation axis.

Advantageously, the invention remains perfectly compatible with, in particular, the mechanical and dimensional IS-1 standard, which enables it to accept without modification all the current and future standardized probes.

### BRIEF DESCRIPTION OF THE DRAWINGS

Further benefits, features and characteristics of the present invention will become apparent to a person of ordinary skill in the art in view of the following detailed description of a preferred embodiment of the invention, made with reference to the annexed drawings, in which like reference characters refer to like elements, and in which:

FIGS. 1 and 2 are elevated perspective views from the top and the bottom respectively, of an insert of a generator connector according to the invention;

FIG. 3 is a front view of the insert of FIG. 1;

FIG. 4 is a cross-section of the insert, taken along line iv-iv of FIG. 3;

FIG. 5 is a cross-section of the insert, taken along line v-v of FIG. 3, in a position able to receive a probe connector;

FIG. 6 is homologous with FIG. 5, with a probe connector introduced into the insert and in position of partial tightening; and

FIG. 7 is homologous with FIG. 5, for a position of maximum tightening.

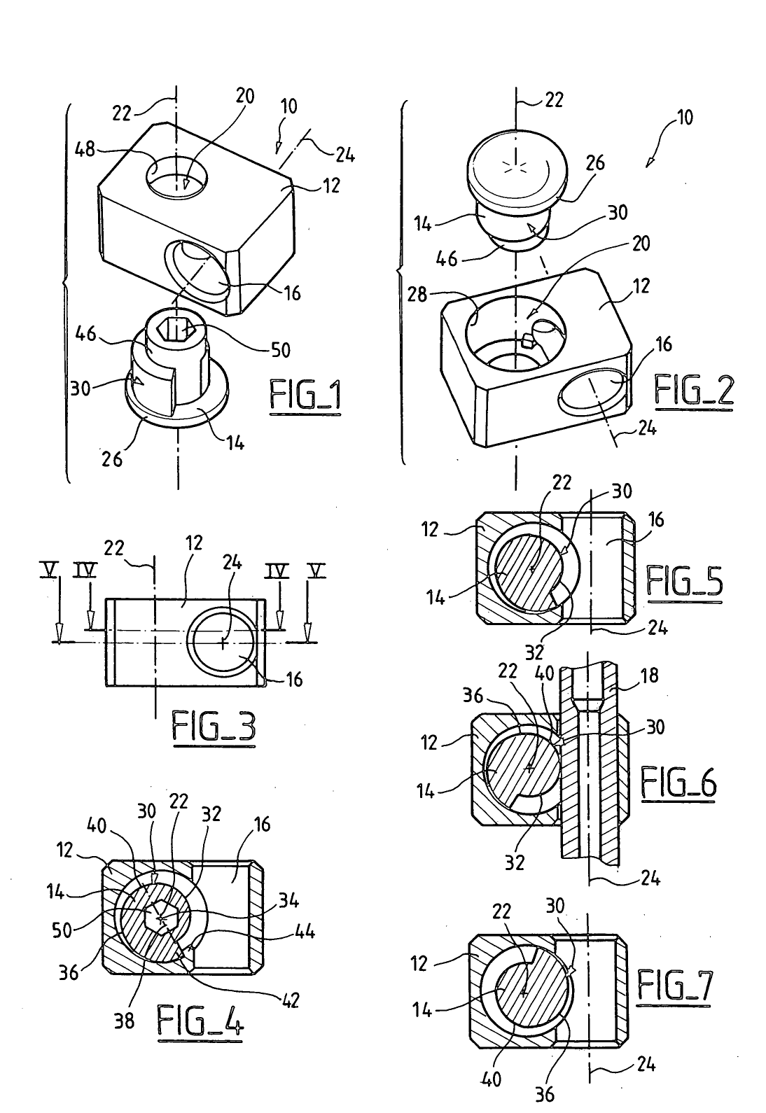

### DETAILED DESCRIPTION OF THE INVENTION

On the figures, reference 10 indicates in a general way an insert for a generator connector system for an active implantable medical device. This connection system insert 10 comprises a housing body 12 and a rotary bolt 14, characteristic of the invention, which will be described hereafter in more detail.

Housing body 12 is the base structure of the insert 10 for a connector of a pulse generator of an active implantable medical device, a massive metal part electrically connected to one of the terminals of the generator by a wire or pin (not represented) and encapsulated in a resin forming the connector head of the device. Housing body 12 includes an axial female housing 16 intended to receive a connector pin 18 (shown in FIG. 6) with which the insert 10 will be mechanically connected, and preferably at the same time electrically connected.

Rotary bolt 14 is laid out in a housing 20 of body 12, having an axis of rotation 22. The bolt 14 and housing 20 are located remotely from the axis 24 of female housing 16, the two axes 22 and 24 preferably being perpendicular to each other. Rotary bolt 14 is guided in rotation around its axis 22 by a peripheral edge of extremity 26 co-operating with the homologous interior edge 28 of cavity 20 in the manner of a smooth bearing.

Rotary bolt 14 has on its intermediate part a side cam surface 30. In the illustrated example (see in particular FIG. 4) cam surface 30 has a first semicylindrical surface 32, whose axis 34 is concentric with bolt rotation axis 22, and a second semicylindrical surface 36, having a larger diameter, whose axis 38 is excentric compared to axis 34. The shift (i.e., the distance) between axes 34 and 38 and the difference between the two diameters of semicylindrical surfaces 32 and 36 are selected as a matter of design choice so that these two semicylindrical surfaces are connected on one side at the extremities (at area 40) forming a smooth transition in continuity and on the opposite side, at area 42, the surfaces are connected with a discontinuity 42. Discontinuity 42 is disposed to come in contact with a bump 44 (FIG. 4) that forms a protuberance inside cavity 20, to limit the travel of the rotary bolt in the counterclockwise direction (with conventions of FIG. 4). The change in diameter of the cam 30, as it rotates about axis 22, is sufficiently large to engage and secure the connector probe inserted in housing 16, as will be discussed.

Lastly, at the axially opposed extremity to the peripheral edge 26, rotary bolt 14 has a cylindrical surface 46 of axis 22, cooperating with an interior surface 48 homologous with cavity 20 so as to also guide in rotation at this place rotary bolt 14 around its axis 22 in the manner of a smooth bearing.

Bolt 22 comprises in addition a drive means structure 50 having a shape complementary to an external tool such as an axial hollow polygonal (e.g., hexagonal) stamp or other shaped cavity 50 allowing its rotation by a complementary shaped drive end of an adapted tool, preferably a nonspecific tool such as a torque limited screwdriver.

The initial configuration of the device of the present invention, i.e., before a probe connector is to be installed, is illustrated in FIGS. 4 and 5. FIG. 4 is a sectional plan view located at a level slightly above the plane containing the axis 24 of female housing 16 and showing bump 44, while the view of FIG. 5 is taken in the plane including the axis 24 of female housing 16. In this initial configuration, the rotary bolt is turned in its retracted position, to its extreme counterclockwise position (as illustrated in FIG. 5), this position being defined and limited by discontinuity 42 coming in contact against bump 44. In this position, the cylindrical part 32 having the smaller diameter is turned in the direction of axial housing 16, and this cam surface does not make a protuberance in the axial female housing 16. Consequently, housing 16 remains free to receive a probe connector.

FIG. 6 illustrates the device after introduction of a probe connector 18 into female housing 16. The clinician, by means of a tool (not shown) introduced into hexagonal stamp 50, has started to turn the rotary bolt 14 in the clockwise direction compared to the initial position of FIG. 4. As the rotational movement is continued, area 40 comes in contact with the external surface of probe connector 18 (FIG. 6). Taking into account the offset between two semicylindrical surfaces 32 and 36, the larger diameter cam surface 36 will gradually protrude inside axial housing 16 because of the offset between axis 38 of the semicylindrical surface of cam 36 and the axis of rotation 22 of bolt 14. This action will exert on the probe connector 18 a force from bolt 14, directed radially, that will secure the probe connector 18 inside female housing 16. The use of a torque limited screwdriver makes it possible to gauge in a precise way the radial force so as to ensure a good mechanical coupling and a satisfactory electric connection, without significant risk of deforming the probe connector.

FIG. 7 illustrates the extreme locking position of rotation of bolt 14, where the projection of the cam surface 30 inside housing 16 is maximal. In practice, this extreme position is not reached if one uses a torque limited screwdriver as a locking position is obtained at the torque limit of the tool before the extreme locking position is reached. The extreme locking position, however, makes it possible to account for some mechanical play between probe connector 18 and housing body 12, as well as possible deformation of probe connector 18 during rotation of the bolt 14, and still obtain a satisfactory mechanical coupling

The following advantages of the device of the present invention are observed to arise from the foregoing description. First, there is a rationalization of production: the stage of installation of the screws can be avoided, and insert 10 having housing body 12 equipped with rotary bolt 14 constitutes a complete substructure that can be assembled in the connector in the same way as a traditional insert, before the molding of the unit. Second, there is a reduction of the volume of the connector, because of the absence of any transversely moving projecting part (more specifically the length of a screw). Third, there is no risk that the screw will fall into the insert, or out of the insert. Fourth, there is a simplicity of implementation for the clinician, with the rotary bolt defining two quite precise positions, namely (i) a retracted position for allowing free insertion of the probe connector (rotation of the bolt/screwdriver counterclockwise to the left) and (ii) a locking position for the tightening of the probe (clockwise rotation of the torque limited screwdriver to the right). The tightening or loosening of the probe connector can be carried out by a rotation with limited race, for example, less than one turn, typically a rotation of the order of a half-turn to three-quarter of turn at the maximum.

In addition, the insert of the present invention allows securing not only cylindrical male structures, which is the most frequent case in probe connections, but, it can also be applied if necessary to the tightening of male structures having a polygonal or other section.

Furthermore, although the invention is described within the framework of a terminal simultaneously ensuring an electric connection and a mechanical coupling, it should be understood that one can dissociate these two functions. Thus, a generator terminal according to the invention can be used only for the mechanical coupling of the extremity of the probe in the generator connector, and the electric connection can be obtained by a separate body or connection.

Suitable devices for which the present invention has application include, for example, the pacemaker, defibrillator and cardiovertor models available from Ela Medical.

One skilled in the art will appreciate that the present invention can be practiced by other than the described embodiments, which are presented for purposes of illustration and not of limitation.

1. A connector system for an active implantable medical device comprising: a generator connector housing body having:
an axial female housing able to receive a probe connector;
a bump; and
a reversible means for mechanical retention of a probe connector in said female housing;
wherein said reversible means comprises a rotary bolt mounted in an aperture in said housing body for rotation about an axis of rotation, said bolt having a drive means structure having a shape complementary to an external tool able to exert on the rotary bolt a rotational driving couple around the rotary bolt axis of rotation, and a side cam surface comprising a first semicircular surface having a first diameter centered on the rotary bolt axis of rotation, and a second semicircular surface having a second diameter greater than the first diameter and an axis of rotation offset with respect to the rotary bolt axis of rotation, said first and second semicircular surfaces being connected in continuity at one of their extremities and in discontinuity at the other of their extremities, said rotary bolt being rotatable in said housing body between a retracted position to permit free movement of the probe connector in said female housing, and a locking position in which the secured semicircular surface of the rotary bolt cam surface protrudes inside the axial female housing so as to exert a radial force on an external surface of a probe connector inserted in said female housing to secure the probe connector inside the female housing, wherein said discontinuity cooperates with said bump to limit the rotation of the rotary bolt in the retracted position.

2. The device of claim 1, wherein said drive means structure shape further comprises a hollow stamp formed axially in the rotary bolt.

3. The device of claim 1, wherein the rotary bolt comprises at a first axial extremity an external cylindrical surface having a third diameter, and wherein the body housing further comprises an internal aperture of said third diameter wherein said rotary bolt external cylindrical structure fits in and cooperates with said body internal aperture cylindrical surface to allow guidance in rotation of the rotary bolt around its axis.
